(19) United States
(12) Patent Application Publication
(10) Pub. No.: US 2013/0245640 A1
(43) Pub. Date: Sep. 19, 2013

(54) DEVICE AND SYSTEM FOR NARROW GAUGE MEDICAL INSTRUMENT NAVIGATION

(71) Applicant: Willet F. Whitmore, III, Longboat Key, FL (US)

(72) Inventor: Willet F. Whitmore, III, Longboat Key, FL (US)

(73) Assignee: CIVCO MEDICAL INSTRUMENTS CO., INC., Kalona, IA (US)

(21) Appl. No.: 13/421,339

(22) Filed: Mar. 15, 2012

(51) Int. Cl. A61B 19/00

(52) U.S. Cl. 606/130

(57) ABSTRACT

A device and system for medical instrument navigation is provided including a handle, a stylet having a distal end, a proximal end and an axially disposed aperture, the proximal end affixed to the handle. A navigation sensor capable of registering positional information is disposed adjacent to the handle for tracking the location of the handle. A pair of thin fibers forming an optic cable is disposed in the aperture, wherein the optic cable measures deflection and the location and direction of the distal end of the stylet relative to the electromagnetic sensor. An interrogator is coupled to the optic cable. The navigation sensor reports its position in space while the interrogator reports the amount and direction of the deflection of the distal end of the stylet in relation to the navigation sensor. A processor analyzes data to determine the location of the distal end of the stylet in space.

### BACKGROUND OF THE INVENTION

The present invention is directed generally to three dimensional navigation systems for medical instruments.

It is desirable in many medical procedures to accurately determine the three dimensional spatial position of the distal tip of a medical device or instrument within a patient. Presently, percutaneous image guided procedures are best accomplished when the target lesion is clearly visible in the three dimensional volume image data and the distal tip of the instrument can be tracked while the stylet of the instrument (e.g., a needle) is advanced to the target. Various technologies have been developed to assist with this approach such as image fusion and electromagnetic tracking with an electromagnetic sensor in the tip of the instrument. Placement of the sensor in the tip is essential in many cases because the tip is often difficult to image and the instruments used in percutaneous procedures tend to be relatively thin and flexible. If the instruments were not (i.e. if the stylet of the instrument was reliably straight and rigid and thus in a fixed relation to the handle), a simpler technological solution with an electromagnetic or optical sensor located on the exterior end of the device can be more cheaply and easily applied.

In some image guided procedures such as in the lung, brain, and thyroid, very thin (small gauge) stylets of instruments are required to keep tissue damage and risk of complications low. In these cases, the physical limits on the size of the electromagnetic sensors disqualifies electromagnetic tracking technology as a solution for tip tracking. Flexibility of the stylet of the instrument makes tracking on the exterior handle end essentially useless. Also, in some larger diameter but still flexible stylets of instruments such as those used for tissue ablation, the entire space within the needle may be taken up by the energy source components such that there remains insufficient space for electromagnetics. A different device and method must be applied to enable instrument tip tracking in these circumstances.

All references cited herein are incorporated herein by reference in their entireties.

### BRIEF SUMMARY OF THE INVENTION

In the present invention, fiber optic technology that measures the relative tip deflection and direction of a stylet portion of an instrument (e.g., a needle) is applied in combination with electromagnetic volume navigation sensors or optical tracking technology during image guided procedures. This added fiber optic technology answers the need for tip tracking in instruments where electromagnetic tracking technology or line of sight optical tracking alone cannot be employed to achieve the desired accuracy.

The present invention uses a very thin (for example, as small as approximately 150 microns in diameter) optic cable having a pair of thin fibers inserted along the full length of the stylet portion of an instrument that is then used to precisely track the amount, location along the stylet, and direction of tip deflection relative to the handle end of the instrument. This information is then combined with the handle that is registered to an electromagnetic sensor capable of registering six degree positional information. The electromagnetic sensor is accurately affixed to the instrument for the purpose of precisely tracking the handle within an electromagnetic field, and thus accurately guiding very delicate and flexible instruments during an image guided procedure.

Presently, similar diameter optical fibers are being used experimentally in a three fiber spaced array typically around the periphery of instruments/needles in combination with other optics to guide instruments during procedures performed in magnetic resonance imaging devices. This application is because electromagnetics cannot be used in magnetic resonance imaging devices. The present invention takes advantage of optical Fiber Bragg Grating technology to bring that same functionality into a pair of fibers which can be placed centrally or peripherally in a linear instrument, and adds utilization with electromagnetic tracking technology to achieve broad application in image guided medical procedures.

In practice, an exemplary system would function as follows:

1) an optic cable having a pair of thin fibers are embedded in the wall or placed in the hollow center of a stylet (e.g., a needle) of an instrument. Alternatively, the optic cable could actually be placed freely in the center or function as the filler of a hollow stylet;

2) beyond the exterior/handle end of the instrument/needle, a coupling connects a reusable Fiber Bragg Grating interrogator to the individual thin fibers of the optic cable.

3) an electromagnetic sensor capable of registering six degrees of positional information is affixed to the handle end of the (preferably disposable) instrument;

4) a sterile cover may protect the reusable fiber optic interrogator, the optic cable may be sterile, and the electromagnetic sensor and a power/communication cable connecting it to the processor or computer that reports the tracking data to the user may be sterile or covered with a sterile sleeve; and

5) the electromagnetic sensor reports the position and orientation of the handle end of the instrument and the distance to the tip inside the patient while the fiber optics reports the amount, location along the stylet and direction of the tip deflection in relation to the electromagnetic sensor.

In this manner, highly-accurate tip tracking can be displayed in real time relative to patient images for virtually any linear instrument in common use for image guided medical procedures.

One preferred embodiment for image guided procedures may be that the optic cable is permanently embedded in the instrument at manufacture and then the assembly is presented to the user in a sterile package for single use. Another preferred embodiment may be that the optic cable is a sterile single cable that is introduced into any appropriate hollow stylet or needle at the time of a procedure. The optic cable may then be removed once the instrument tip is in the desired position to complete a procedure. The electromagnetic sensor may also be pre-attached to the instrument or optic cable at the handle end, or independently, removably clipped on to the handle end of the instrument, as desired. The configuration at the handle end of the instrument may allow sterile coupling to a reusable optic cable that is connected to the interrogator, or the optic cable may be continuous from inside the instrument to the interrogator. Alternatively, a pre-sterilized length of fiber optic cable may be plugged into the handle that already includes the electromagnetic sensor. The optic cable may be inserted into any standard hollow needle assembly and secured via a Luer lock connection. The fiber will be inserted along a sufficient length to accurately track the stylet of the instrument's tip. For procedures where the additional costs can be justified, factory assembly and sterilization of all components may be done for single use.

In accordance with the present invention, a device for narrow gauge medical instrument navigation is provided. The device includes a handle and a stylet (such as a needle) that may have a closed or open end. The stylet has a distal end, a proximal end and an axially disposed aperture extending from the distal end to the proximal end. The proximal end is affixed to the handle. A navigation sensor is disposed adjacent to and in fixed relation to the handle wherein the navigation sensor is capable of registering six degree positional information for tracking the location of the handle in space. The distal end of the stylet is at a known location relative to the navigation sensor when the stylet is in an unflexed condition. A pair of thin fibers forming an optic cable is disposed in the aperture of the stylet and is disposed in fixed relation to the navigation sensor. The pair of thin fibers (preferably with Fiber Bragg Grating) form the optic cable that is disposed substantially entirely from the distal end to the proximal end. The optic cable is adapted to measure deflection and the location and direction of deflection of the stylet relative to the electromagnetic sensor.

The navigation sensor may be an electromagnetic sensor, an optical tracking sensor, or any other type of sensor capable of establishing its position in three dimensional space. The optic cable containing the pair of thin fibers may be substantially any suitable shape having a maximum diameter of about 300 microns or less. A very thin fiber optic cable of approximately 150 microns to 300 microns in diameter is preferable. The fiber optic sensor preferably utilizes Fiber Bragg Grating technology.

In one exemplary embodiment, the stylet may be a needle. The optic cable may be placed in the aperture or otherwise disposed in the aperture such that it is in a fixed position, or embedded at manufacture in the wall of the stylet. A sterile cover may be provided to protect the navigation sensor or any re-usable cables, as required, to develop and maintain a sterile field. The optic cable may be removable by a user during an image guided procedure. The navigation sensor may be removably disposed on the handle.

A system for medical instrument navigation is also provided. The system includes the device above, an interrogator coupled to the optic cable in the instrument, and a processor to analyze data from the interrogator and the navigation sensor to determine the precise location of the distal end of the stylet in a defined three dimensional space. The navigation sensor is for reporting its position and orientation in three dimensional space while the interrogator is for reporting the amount, location and direction of the deflection of the distal end of the stylet in relation to the navigation sensor that is in a known and fixed relation to the handle of the instrument. A display for displaying the orientation of the instrument, and/or a position of the distal end of the stylet in a body of a being may also be provided. The interrogator and the processor may be an integral unit.

### BRIEF DESCRIPTION OF SEVERAL VIEWS OF THE DRAWINGS

The invention will be described in conjunction with the following drawings in which like reference numerals designate like elements and wherein:

FIG. 1 is a schematic view of a device for narrow gauge medical instrument navigation in accordance with an exemplary embodiment of the present invention;

FIG. 2 is an isometric view of an exemplary device for narrow gauge medical instrument navigation of FIG. 1;

FIG. 3 is a s cross sectional view of the device for narrow gauge medical instrument navigation of FIG. 2, taken substantially along lines III-III of FIG. 2;

FIG. 4 is a cross sectional view of a stylet of the device for narrow gauge medical instrument navigation of FIG. 2, shown at detail 4 of FIG. 3;

FIG. 4A is a cross sectional view of the stylet of FIG. 4, taken substantially along lines IVa-IVa of FIG. 4;

FIG. 5 is a simplified schematic diagram of a system for determining the three dimensional position of the distal end of a stylet in the device for narrow gauge instrument navigation of FIG. 1; and

FIG. 6 is side, cross-sectional view of the device of FIG. 2, shown in sterile packaging.

### DETAILED DESCRIPTION OF THE INVENTION

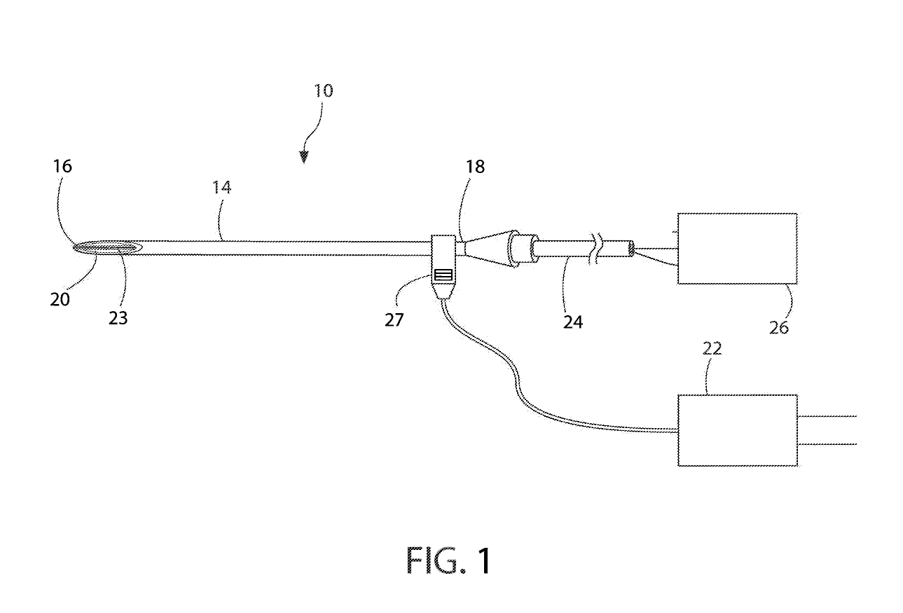

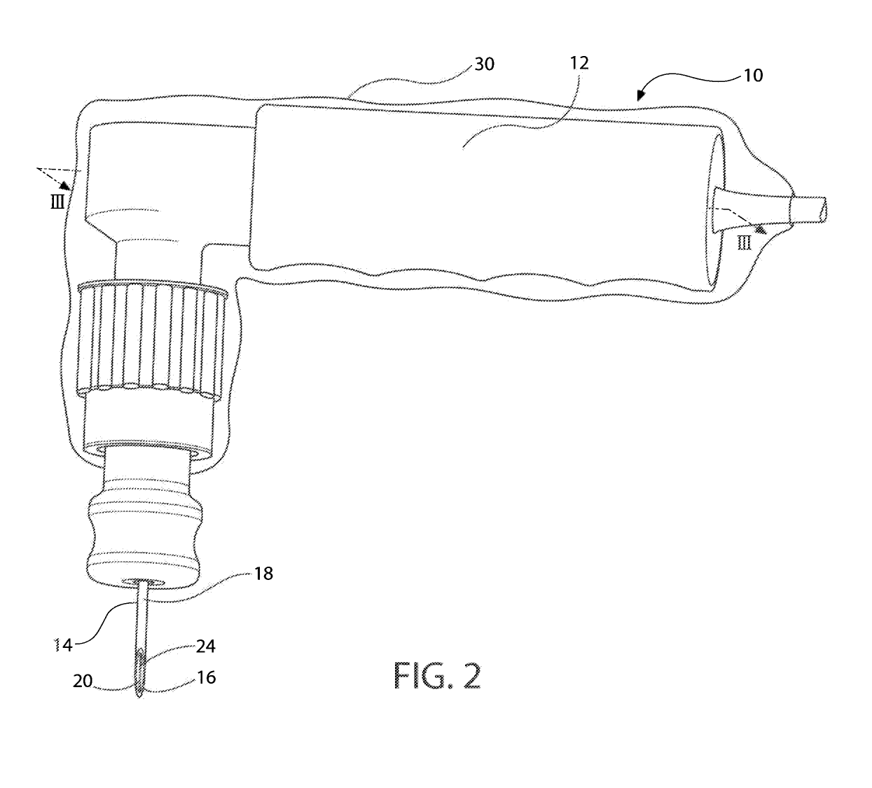

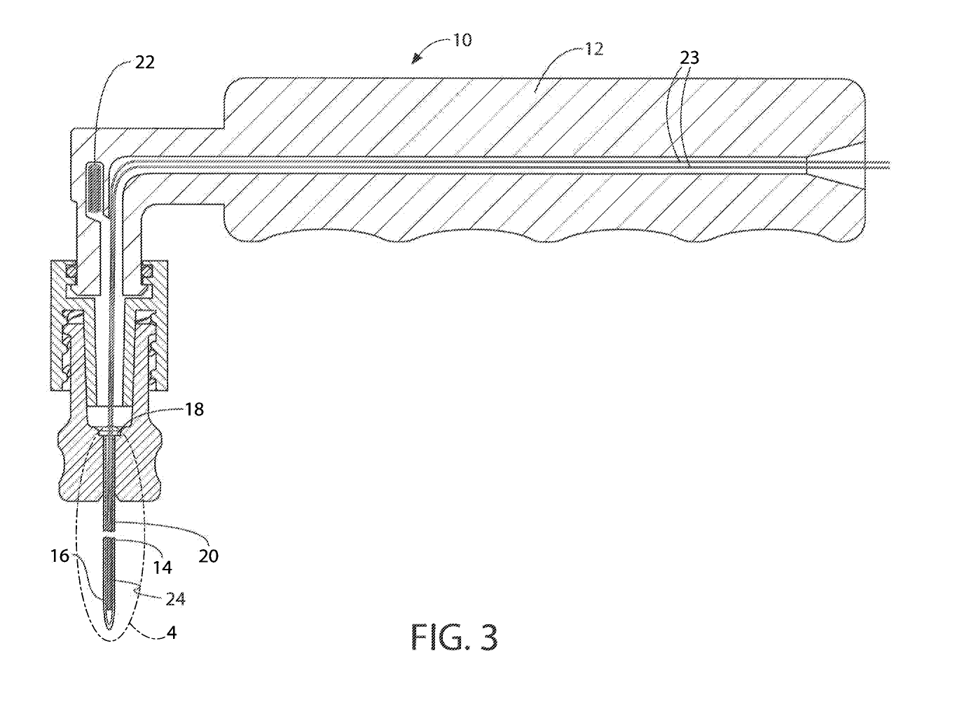

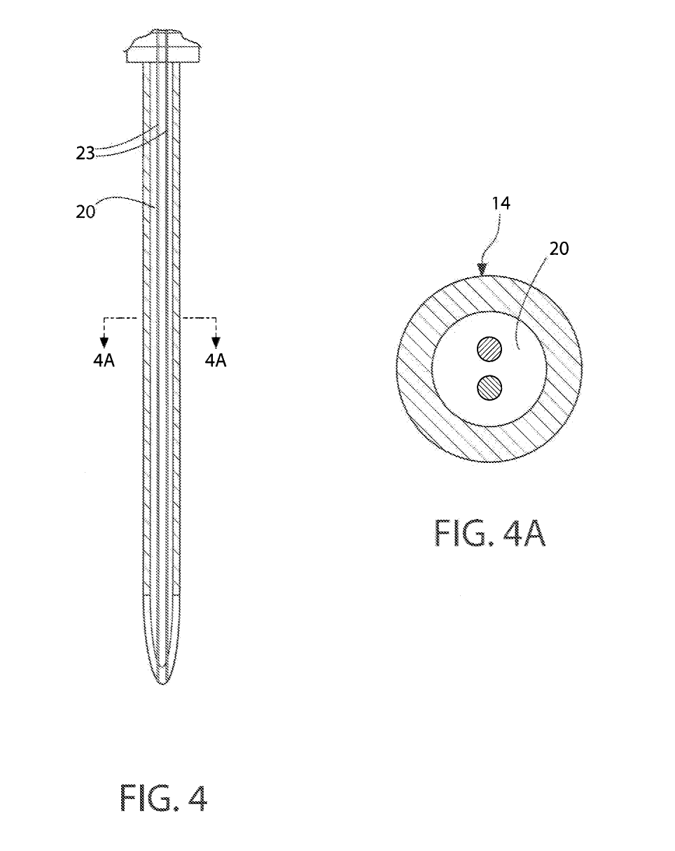

The invention will be illustrated in more detail with reference to the following embodiments, but it should be understood that the present invention is not deemed to be limited thereto. Referring now to the drawing figures, wherein like part numbers refer to like elements throughout the several views, there is shown in FIGS. 1 and 2 a device for narrow gauge medical instrument navigation 10 in accordance with an exemplary embodiment of the present invention. The device includes a handle 12, a stylet 14 (such as a needle, as shown) having a distal end 16, a proximal end 18 and an axially disposed aperture 20 extending from the distal end 16 to the proximal end 20. The proximal end 20 of the stylet 14 is affixed to the handle 12.

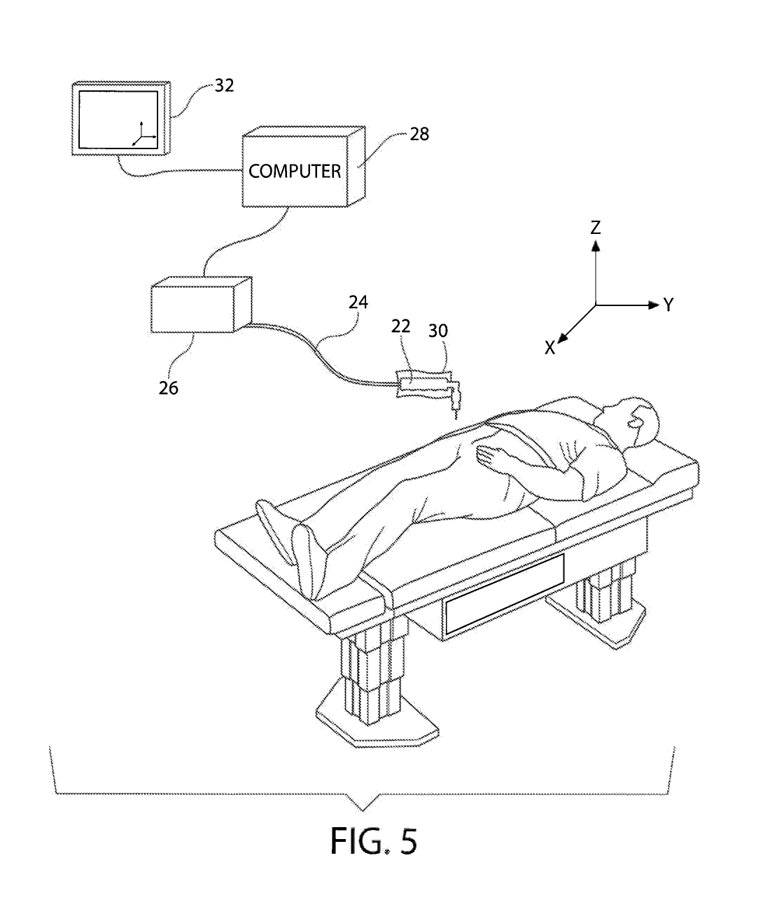

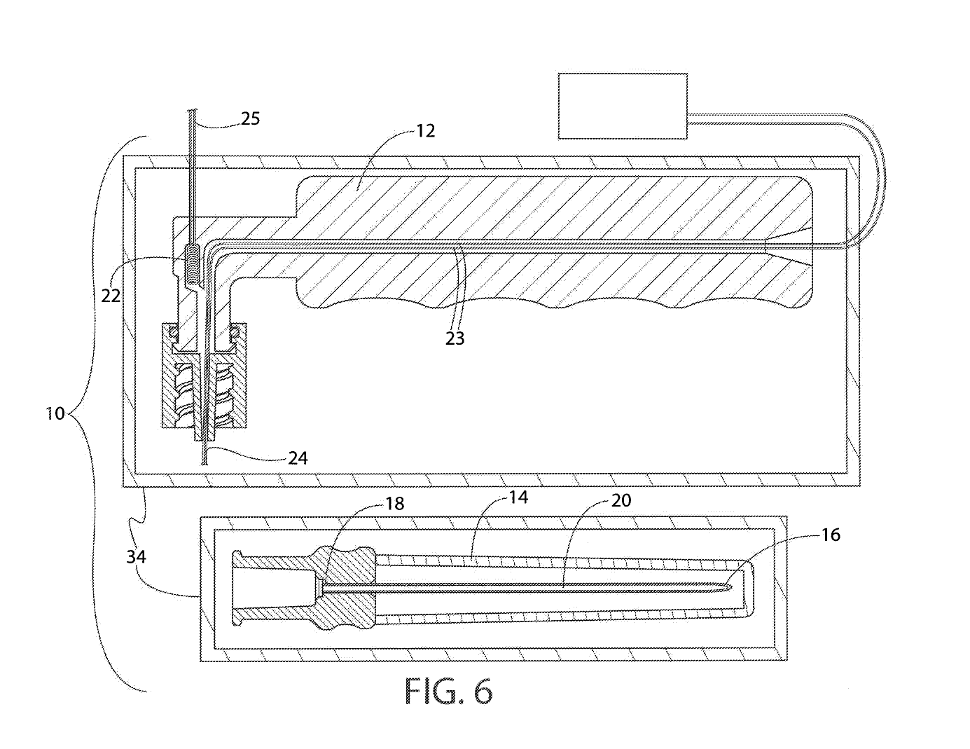

A navigation sensor 22 (such as an electromagnetic sensor, an optical tracking sensor, or similar) is disposed adjacent to the handle 12. The navigation sensor 22 is preferably capable of registering six degree positional information for tracking the location of the handle in space. The distal end 16 of the stylet 14 is at a known location from the navigation sensor 22 when the stylet is in an unflexed condition. The navigation sensor 22 may be connected by a cable 25 that provides power from a power supply 27 and also serves as a data cable. See FIG. 5. A pair of thin fibers 23 form an optic cable 24 that is disposed in the axially disposed aperture 20 of the stylet 14 and is disposed in fixed relation to the navigation sensor 22 (for example, located in the aperture 20 or embedded in the aperture). The optic cable is preferably disposed substantially entirely from the distal 16 end to the proximal end 18 of the stylet 14. The optic cable 24 is adapted to measure deflection and the location and direction of the distal end 16 of the stylet 14 relative to the navigation sensor 22.

An interrogator 26 (such as a Fiber Bragg Grating interrogator) is coupled to the optic cable 24. The thin fibers 23 that form the optic cable have light attenuation characteristics which vary in accordance with the direction and flex of the stylet 14, for example, Fiber Bragg Grating, in the portion of the optic cable 24 located in the stylet. See, for example, U.S. Pat. No. 7,903,907 (Park et al.). The interrogator 26 analyzes this data to provide precise positional data in space. The navigation sensor 22 reports its position and orientation in three dimensional space while the interrogator 26 reports the amount and direction of the deflection of the distal end 16 of the stylet 14 in relation to the navigation sensor 22. A processor 28 analyzes data from the interrogator 26 and the navigation sensor 22 to determine the precise location of the distal end 16 of the stylet 14 in three dimensional space. The processor is preferably connected to a display 32 to display to the physician or other user of the system the precise location of the needle within a patient.

It is typically desirable that the fiber optic cable be as thin as possible, for example, approximately 150 microns to 300 microns in diameter. A sterile cover 30 may be provided to protect any element or elements of the device 10, including the navigation sensor 22.

While the invention has been described in detail and with reference to specific embodiments thereof, it will be apparent to one skilled in the art that various changes and modifications can be made therein without departing from the spirit and scope thereof.

What is claimed is:

1. A device for narrow gauge medical instrument navigation, comprising:
(a) a handle;
(b) a stylet having a distal end, a proximal end and an axially disposed aperture extending from the distal end to the proximal end, the proximal end affixed to the handle;
(c) a navigation sensor disposed adjacent to said handle and in fixed relation to said handle wherein the navigation sensor is capable of registering six degree positional information for tracking the location of the handle in space, the distal end of the stylet being at a known location from the navigation sensor when the stylet is in an unflexed condition; and
(d) a pair of thin fibers forming an optic cable disposed in the aperture of the stylet disposed in fixed relation to the navigation sensor and disposed substantially entirely from said distal end to said proximal end, wherein the optic cable is adapted to measure deflection and the location and direction of deflection of the stylet so as to track the distal end of the stylet relative to the navigation sensor.

2. The device for narrow gauge medical instrument navigation of claim 1, wherein the navigation sensor is an electromagnetic sensor.

3. The device for narrow gauge medical instrument navigation of claim 1, wherein the navigation sensor is an optical tracking sensor.

4. The device for narrow gauge medical instrument navigation of claim 1, wherein the optic cable is approximately 150 microns to 300 microns in maximum cross-sectional width.

5. The device for narrow gauge medical instrument navigation of claim 1, wherein the optic cable and interrogator utilize Fiber Bragg Grating technology.

6. The device for narrow gauge medical instrument navigation of claim 1, wherein the stylet is a needle.

7. The device for narrow gauge medical instrument navigation of claim 1, wherein the thin fibers that form the optic cable are embedded in the wall of the stylet

8. The device for narrow gauge medical instrument navigation of claim 1, wherein the fibers are assembled in the optic cable, wherein the optic cable is insertable into the stylet, lockable into a fixed position in the stylet, and removable at will from the stylet.

9. The device for narrow gauge medical instrument navigation of claim 1, including a sterile cover to protect the navigation sensor.

10. The device for narrow gauge medical instrument navigation of claim 1, wherein the optic cable is removable by a user during an image guided procedure.

11. The device for narrow gauge medical instrument navigation of claim 1, wherein the navigation sensor is removably disposed on the handle.

12. A system for medical instrument navigation, comprising:
(a) a device for narrow gauge medical instrument navigation, comprising:
(i) a handle;
(ii) a stylet having a distal end, a proximal end and an axially disposed aperture extending from the distal end to the proximal end, the proximal end affixed to the handle;
(iii) a navigation sensor disposed adjacent to said handle wherein the navigation sensor is capable of registering six degree positional information for tracking the location of the handle in space, the distal end of the stylet being at a known location from the navigation sensor when the stylet is in an unflexed condition;
(iv) a pair of thin fibers forming an optic cable disposed in the aperture of the stylet disposed in fixed relation to the navigation sensor and disposed substantially entirely from said distal end to said proximal end, wherein the fiber optic cable is adapted to measure deflection and the location and direction of deflection of the shaft so as to track the distal end of the stylet relative to the navigation sensor;
(b) an interrogator coupled to the optic cable to analyze data received from the optic cable;
(c) a processor to analyze data from the interrogator and the navigation sensor to determine the precise location of the distal end of the stylet in three dimensional space; and
(d) wherein the navigation sensor is for reporting its position and orientation in three dimensional space while the interrogator is for reporting the amount and location and direction of the deflection of the stylet so as to track the distal end of the stylet in relation to the navigation sensor.

13. The system of claim 12, wherein the navigation sensor is an electromagnetic sensor.

14. The system of claim 12, wherein the navigation sensor is an optical tracking sensor

15. The system of claim 12, wherein the fiber optic cable is approximately 150 microns to 300 microns in maximum cross sectional width.

16. The system of claim 12, wherein the fiber optic sensor and interrogator utilize Fiber Bragg Grating technology.

17. The system of claim 12, wherein the stylet is a needle.

18. The system of claim 12, wherein the optic cable is embedded in the aperture.

19. The system of claim 12, including a display for displaying a position of the distal end of the stylet in a body of a being.

20. The system of claim 12, including a sterile cover to protect the navigation sensor.

21. The system of claim 12, wherein the interrogator and the processor are an integral unit.
